(19) United States
(12) Patent Application Publication
(10) Pub. No.: US 2015/0364861 A1
(43) Pub. Date: Dec. 17, 2015

(54) IMPLANTABLE CONNECTION MECHANISMS FOR CONTINUOUS HIGH POWER DELIVERY

(71) Applicant: Minnetronix, Inc., St. Paul, MN (US)

(72) Inventors: Lori Lucke, Rosemount, MN (US); David John Hansen, Stillwater, MN (US)

(21) Appl. No.: 14/706,714

(22) Filed: May 7, 2015

Related U.S. Application Data
(60) Provisional application No. 62/013,245, filed on Jun. 17, 2014.

(51) Int. Cl. H01R 13/621; H01R 13/52; A61M 1/10

(52) U.S. Cl. CPC H01R 13/621; A61M 1/1008; H01R 13/5219

(57) ABSTRACT

A connection mechanism for an implantable system. The implantable system may include an implantable device, such as a mechanical circulatory device, an implantable converter or control system, and an implantable power system, such as a transcutaneous energy transfer system. The connection mechanism may include a connector connected to one of the implantable device or the power system by wires. The connection mechanism may include a mating receptacle configured at the control system for receiving the connector. The connection mechanism may further include a retention mechanism. The retention mechanism may provide mechanical and/or electrical connection between the connector and the receptacle. The connector, the receptacle, and the retention mechanism may be configured to establish continuous high power or high current electrical communication between the control system and one of the implantable device or the power system.

### CROSS-REFERENCE TO RELATED APPLICATIONS

This application claims priority to U.S. Provisional Application No. 62/013,245, entitled “Implantable Connection Mechanisms for Continuous High Power Delivery,” filed Jun. 17, 2014, which is incorporated by reference in its entirety herein.

### TECHNOLOGICAL FIELD

The present disclosure generally relates to implantable connection mechanisms. More particularly, the present disclosure relates to implantable connection mechanisms for continuous high power delivery.

### BACKGROUND

Implantable medical devices such as heart pumps, ventricular assist devices, and the like may operate under high voltage, high current, current surges, and/or continuous power consumption conditions and thus may tend to use large amounts of power. To improve the quality of life for the patient receiving such high power implantable devices, it may be desirable to implant certain system components that control the operation of the medical device and system components, such as portions of a transcutaneous energy transmission system, that provide electrical power. Connection mechanisms such as cables may also be implanted that operably interconnect the implantable device, the control system, the power system, and so on.

Existing implantable connection mechanisms have been designed for low power implantable devices or for devices that only momentarily deliver a high pulse of energy. An aspect of high power consuming implantable devices, such as heart pumps, is that these devices run continuously to support the patient's life. It is therefore desirable to provide implantable connection mechanisms for continuous high power delivery that securely connect the various components and that can carry large currents and/or voltages continuously with minimal parasitic losses or degradation.

### SUMMARY

The present disclosure therefore provides various connection mechanisms. In particular, this disclosure describe a connection mechanism for use in implantable systems having an implantable device, such as mechanical circulatory devices, heart pumps, and so on, which may require continuous high power supply. In some implementations, the implantable system may include an implantable device, an implantable power converter or control system, and an implantable power system. The connection mechanism may include at least one connector, at least one mating receptacle, and at least one retention mechanism. The connector may be operably coupled to one of the implantable device or the power system. The receptacle may be at least partially hermetically sealed within the control system. The retention mechanism may provide a mechanical and/or electrical connection between the connector and the receptacle for continuous high power delivery between the control system and at least one of the implantable device or the implantable power system.

In a first aspect, the present disclosure is directed to a connection mechanism for an implantable system, comprising a connector, a receptacle configured for receiving the connector to establish high power or high current electrical communication between the connector and the receptacle, and a retention mechanism for mechanically and/or electrically engaging the connector within the receptacle.

In some implementations, the implantable system further comprises an implantable device, an implantable converter, and an implantable power system, where the connection mechanism is configured to establish high power or high current electrical communication between the converter and one of the implantable device or the power system.

In some implementations, the power system comprises an implantable transcutaneous energy transfer system.

In some implementations, the implantable device comprises a mechanical circulatory device.

In some implementations, the receptacle is at least partially hermetically sealed within a header portion of the converter.

In some implementations, the connector comprises a circumferential groove having two annular side wall portions and a cylindrical base portion between the side wall portions, and the retention mechanism is configured to engage the groove by engaging at least one of the side wall portions or the base portion.

In some implementations, the retention mechanism comprises a retention clip including two arms each configured to engage a surface portion of the groove of the connector and having a protrusion formed at the free end thereof.

In some implementations, the retention mechanism comprises a retention key oriented perpendicularly to the longitudinal extension of the connector.

In some implementations, the retention mechanism comprises a wire looped around the groove of the connector.

In some implementations, the retention mechanism comprises a compression-biased mechanism.

In some implementations, the compression-biased mechanism is biased against the groove of the connector by a spring member.

In some implementations, the compression-biased mechanism at least partially retracts into a recess formed within the receptacle when the connector is being inserted into the receptacle.

In some implementations, the compression-biased mechanism comprises one or more of a pin member, a shell member, and a pair of clamp members operably coupled to a spring member.

In some implementations, the retention mechanism comprises an elastic protrusion extending from the receptacle, where the elastic protrusion is configured to engage the groove of the connector by being at least partially received in the groove of the connector.

In some implementations, the retention mechanism may include a spring/compression member configured around the groove of the connector.

In some implementations, the spring/compression member comprises a spring.

In some implementations, the spring/compression member comprises at least one leaf spring.

In some implementations, the retention mechanism comprises a plurality of elastomeric sealing members arranged at the receptacle, the elastomeric sealing members configured to engage the connector by friction or surface contact between the elastomeric sealing member and the connector.

In some implementations, the retention mechanism comprises a fastener configured to engage a tip portion of the connector through an aperture formed at an end portion of the receptacle.

In some implementations, the fastener comprises one of a collet, a screw, or a bolt.

In some implementations, the fastener and/or the tip portion of the connector may be biased against the end portion of the receptacle.

In some implementations, the retention mechanism comprises a cotter pin configured to engage at least one through hole formed at the connector.

This summary of the disclosure is given to aid in the understanding of the disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The present invention will now be described by way of example only with reference to the following figures in which:

FIG. 1 is a schematic illustration of an implantable system having an implantable device, an implantable control system, and an implantable power system.

FIG. 2 is a schematic illustration of a connection mechanism according to one example for coupling an implantable device to an implantable control system.

FIG. 3 is a schematic illustration of a connection mechanism according to one example for coupling an implantable power system to an implantable control system.

FIGS. 4A-4T are schematic illustrations of retention mechanisms for providing mechanical and/or electrical coupling within the connection mechanism.

The structures in the figures are intended to illustrate and aid in the understanding of the invention, and as such are not intended to necessarily reflect proportions or dimensions.

### DETAILED DESCRIPTION

Aspects of the present disclosure include cables, connectors, and securement mechanisms for an implantable system. The implantable system may include a medical device, such as a mechanical circulatory device, that is implanted within a subject. The implanted medical device may be configured to receive electrical power from one or more power sources having components which are wholly or partially implanted within the subject and/or which are externally located. The implanted system may include a converter and a controller or other component disposed between the implanted medical device and the power source that operates to convert power output from the power source into a form that is usable by the implanted medical device. The cables, connectors, and securement mechanisms of the present disclosure are configured to safely and securely transfer electrical power between the power source, converter, implanted medical device, and/or other components of the implantable system.

A connection mechanism in accordance with this disclosure may include a connector disposed (e.g., at the end of a wire) that provides a connection to a component of the implantable system. The connection mechanism may further include a mating receptacle associated with a component of the implantable system and configured to receive the connector. The connection mechanism may further include a retention mechanism associated with the connector and/or receptacle. The retention mechanism may provide mechanical and/or electrical connection between the connector and the receptacle. The connector, the receptacle, and the retention mechanism may be configured to establish high voltage or high current electrical communication between components of the implantable system for power delivery.

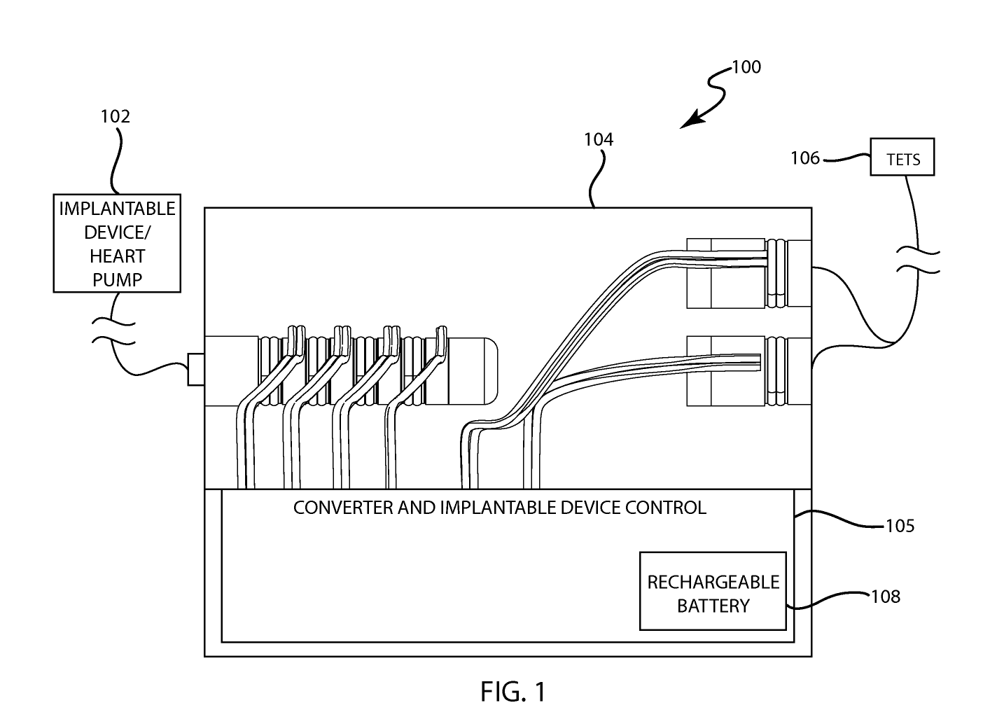

FIG. 1 is a block diagram of an implantable system that includes components and features in accordance with the present disclosure. The implantable system, which is generally referred to with reference number 100, may include an implantable device 102. The implantable device 102 may be any medical device capable of being implanted in a subject, such as a heart pump, an artificial heart, a right ventricular assist device, a left ventricular assist device, a BIVAD, a minimally invasive circulatory support system, a cardiac pacemaker, and so on. While the implanted device 102 may be any implantable medical device, this disclosure describes the implantable system 100 in the context of a heart pump by way of example and not by way of limitation.

The implanted medical device 102 may be configured to receive electrical power from one or more power sources having components which are wholly or partially implanted within the subject and/or which are externally located. In some implementations, the implanted medical device 102 receives electrical power that is wirelessly transmitted through the skin of the subject through the operation of a transcutaneous energy transfer system (TETS) 106. The transcutaneous energy transfer system 106 may include a primary resonant network that is located externally from the subject and a secondary resonant network that is implanted within the subject. The primary and secondary resonant networks may include inductive coils so as to together form a loosely coupled transformer, with the external coil acting as a primary winding and the internal coil acting as a secondary winding. The coils and capacitors associated with the coils may be connected to form a resonant circuit. The coils may be tuned to the same or different resonant frequencies. For example, the coils may be series tuned to a power transmission frequency of about 200 kHz. The external coil may be driven with an alternating current which induces a corresponding electric current in the internal coil due to the coupling between the coils. The current induced in the internal coil can then be used to provide electrical power for the implanted medical device 102 or other components of the implanted system 100.

One or more of the implantable medical device 102 and the transcutaneous energy transfer system 106 may connect to each other through a header 104 that forms a portion of a converter, controller, and/or other component 105 of the implantable system 100. In some implementations, the header 104 forms a portion of a converter that is disposed between the implanted medical device 102 and the transcutaneous energy transfer system 106 and that is configured to convert power output from transcutaneous energy transfer system 106 into a form that is usable by the implanted medical device 102. Here, the converter may first receive alternating current from the transcutaneous energy transfer system 106 at a frequency that is a function of the resonant frequency of the resonant circuit that is associated with the transcutaneous energy transfer system 106. The converter may then convert the electric energy from this alternating current into a form that is usable by the implanted medical device 102, which in some implementations includes a three-phase motor. In some implementations, the header 104 forms a portion of controller or control system that includes processing units and circuitries for controlling the operation of the implantable device 102 or other portions of the implantable system 100.

In some implementations, the converter or controller component 105 may be configured with an implanted battery 108. The implantable battery 108 may be configured to provide power to the implanted medical device 102 when power is not available from the transcutaneous energy transfer system 106, and implantable battery 108 may be rechargeable. For example, during certain time periods, the subject may be located away from the external resonant network portion of the transcutaneous energy transfer system 106 or the external network may be unavailable for other reasons. Here, the implanted system 100 may switch to receive electrical power from the battery 108 so as to maintain an uninterrupted supply of electrical power to the implanted medical device 102. The implanted battery 108 may be rechargeable and, in some embodiments, may be recharged by electrical power transfer received through the operation of the transcutaneous energy transfer system 106.

The implantable device 102, the transcutaneous energy transfer system 106, and/or the header 104 may be coupled through connection cables and connection mechanisms for electrically connecting and mechanically securing the ends of the connection cables to the respective device or systems for power delivery, such as continuous high power delivery. By way of example, FIG. 1 shows connection mechanisms within the header 104 for connection to the implantable device 102 and the transcutaneous energy transfer system 106. The cables, connectors, and securement mechanisms of the present disclosure are described with the reference to these example header 104 connections. It should be appreciated, however, that the cables, connectors, and securement mechanisms of the present disclosure may additionally be used in connection with other components of an implantable system 100, such as the implantable device 102, the transcutaneous energy transfer system 106, and so on.

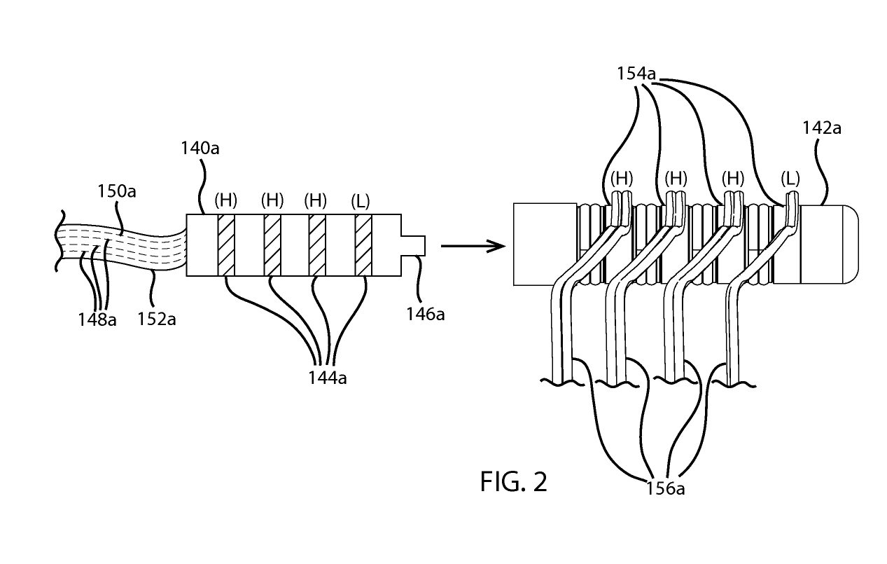

With reference to FIG. 2, one example of a connection mechanism for coupling the implantable device 102 to the header 104 will be described. The implantable device 102 may include a mechanical circulatory device having a three-phase motor. The connection mechanism may include a connector 140a connected to the implantable device 102 and a mating receptacle 142a configured at the header 104. The connector 140a may include four pole connections 144a (or ring or other connections). In some implementations, the connector 140a may include a tip electrode 146a. Insulation may be provided between each pole connection 144a as well as to the tip electrode 146a. The four pole connections 144a may include three pole connections 144a configured to support high current connections and the fourth pole connection 144a configured to support a ground or case connection to the implantable device 102 (e.g., represented in FIG. 2 as “(H)” and “(L)”). The connector 140a may be connected to the implantable device 102 with three high current wires 148a and one low current wire 150a. The wires 148a, 150a may be insulated and sealed to form a single cable 152a. The low current connection may be used as a shield around the high current connections within the cable 152a. One end of the cable 152a is connected to the implantable device 102. The other end of the cable 152a is connected to the connector 140a. The connector 140a may further include sealing mechanisms configured near the cable end (or the distal end) of the connector 140a.

The mating receptacle 142a may be configured within a hermetically sealed header 104 of a converter or controller component 105 of the implantable system 100. The receptacle 142a may include four electrical contacts 154a that may be configured to support three high current connections for connection to an internal circuitry of the converter or controller 105 associated with the header and one low current connection for connection to the ground or the case of the header 104 via wired connections 156a. The wired connections 156a may provide contact from the receptacle 142a to power or control circuitry through a hermetically sealed feedthrough. The wired connections 156a may include three wired connections configured to support high current and one wired connection configured to support low current. Insulation and sealing may be provided between each contact in the header 104 of the converter or controller 105. It is to be appreciated that other phase motors (e.g., four phase, etc.) may be used, and that the number of contacts, etc. may vary depending on the desired application.

When the connector 140a is received within the receptacle 142a, the three high current connections of the connector 140a may be coupled to, be connected to, or mate with the three high current connections of the receptacle 142a and the low current connection may be coupled to, be connected to, or mate with the low current connection of the receptacle 142a. To secure the coupling/connection between the corresponding high/low current connections 144a, 154a of the connector 140a and the receptacle 142a, an additional securement mechanism, such as a mechanical retention mechanism, may be used. Such a mechanical retention mechanism may also provide electrical contact in addition to mechanical retention. The tip electrode 146a may also be connected to a receiving portion of the receptacle 142a using a similar retention mechanism for mechanical retention. The retention mechanisms (described in more detail below) may include set screws, spring contacts, and so on.

The connector and receptacle are generally described herein as having four pole connections, but it should be appreciated that alternative configurations may include a greater or lesser number of pole connections with both low current and high current connections. In some examples, the connector may include three pole connections (or ring connections) and a tip electrode. Insulation may be provided between each electrical contact of the connector. The three pole connections may be configured to support high current connections and the tip electrode may be configured to support a ground or case connection to the implantable device 102. The connector may be connected to the implantable device 102 with three high current wires and one low current wire. The wires may be insulated and sealed to form a single cable. The low current connection may be used as a shield around the high current connections within the cable. One end of the cable is connected, directly or through a connector, to the implantable device 102. The other end of the cable is connected to the connector. The connector may further include sealing mechanisms configured near the cable end (or the distal end) of the connector.

When the connector includes three pole connections (or ring connections) and a tip electrode, the mating receptacle may be configured with electrical contacts to support three high current connections for connection to the internal circuitry of the converter or controller 105 associated with the header 104 and to support ground connection for connection to the ground or the case of the header 104 via wired connections. The wired connections may provide contact from the receptacle to power or control circuitry through a hermetically sealed feedthrough. The wired connections may include three wired connections configured to support high current and one wired connection configured to support low current. Insulation and sealing may be provided between each contact in the header 104 of the converter or controller 105.

Similar to the example described with reference to FIG. 2, when the three pole connection plus tip electrode connector is received within the receptacle, the three high current connections of the connector may be coupled to, be connected to, or mate with the three high current connections of the receptacle. The tip electrode may be may be coupled to, be connected to, or mate with the ground or other connection of the receptacle. Retention mechanisms, such as set screws, spring contacts, and so on, may be used to secure the coupling/connection between the receptacle and the high current connections and the tip electrode of the connector. The retention mechanisms may provide mechanical retention and/or electrical contact.

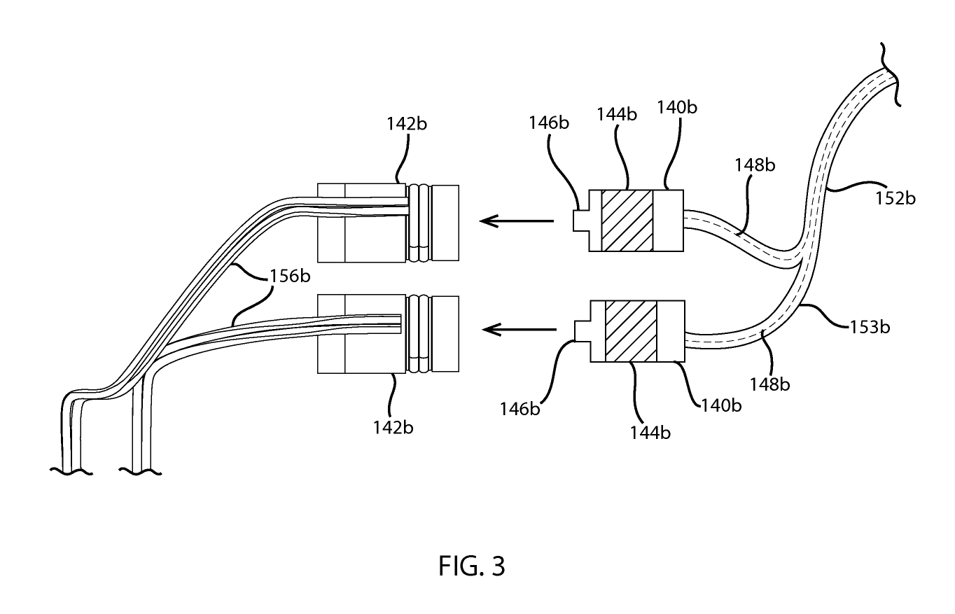

With reference to FIG. 3, one example of a connection mechanism for coupling the implantable TETS 106 to the header 104 will be described. The connection mechanism may include two connectors 140b for connection to the TETS coil and two corresponding receptacles 142b configured at the header 104. Each of the connectors 140b may include a single pole connection 144b (or ring connection) and a tip electrode 146b. Each single pole connection 144b may be configured to support high current. Insulation may be provided between each pole connection 144b on the connector 140b and the tip electrode 146b. The two connectors 140b may be connected to the TETS coil via two high current wires 148b. The wires 148b are insulated and sealed and form a single cable 152b. One end of the single cable 152b is connected to the TETS coil. The other end of the single cable 152b may be configured with a bifurcation 153b to support connection to the two single pole connectors 140b. Each of the single pole connectors 140b may further include sealing mechanisms configured near the cable end (or the distal end) of the connector 140b.

The two mating receptacles 142b may be configured within the hermetically sealed header 104 of the converter or controller 105 for receiving electrical power from the TETS 106 component. The two receptacles 142b may each be configured to support a high current connection via wired connections 156b for connection to the internal circuitry of the converter or controller 105 associated with the header 104. The wired connections 156b may provide contact from the receptacles 142b to the power or control circuitry through a hermetically sealed feedthrough. Insulation and sealing may be provided between each contact in the header 104 of the implantable converter or controller 105.

In some examples, instead of using two connectors each configured with a single pole connection and a tip electrode, one single connector configured with two pole connections (or ring connections) and a tip electrode may be used for coupling the TETS coil to the header 104. The two pole connections may be configured to support high current. Insulation may be provided between each pole connections as well as to the tip electrode. The connector may be connected to the TETS coil with two high current wires. The wires are insulated and sealed and form a single cable. One end of the cable is connected to the TETS coil. The other end of the cable is connected to the two-pole connector. The two-pole connector may further include sealing mechanisms configured near the cable end (or the distal end) of the connector. Other numbers of poles and connectors may be used.

The converter or controller 105 may be configured with a single receptacle for coupling with the two-pole connector. The receptacle may be received within the hermetically sealed header 104 of the converter or controller 105. The mating receptacle may be configured with electrical contacts to support two high current connections for connection to the internal circuitry of the converter or the controller 105 via wired connections. The wired connections may provide contact from the receptacle to the power or control circuitry through a hermetically sealed feedthrough. Insulation and sealing may be provided between each contact in the header 104 of the implantable converter or controller 105.

Similar to the example described with reference to FIG. 3 when the two pole connection plus tip electrode connector is received within the receptacle, the two high current pole connections of the connector may be coupled to, be connected to, or mate with two high current connections of the receptacle. The tip electrode may be coupled to, be connected to, or mate with a receiving portion of the receptacle. Retention mechanisms, such as set screws, spring contacts, and so on, may be used to mechanically and/or electrically couple/connect the receptacle and the high current connections.

Although the connectors 140a, 140b as described above are configured with a tip electrode 146a, 146b, it is not required for each connector to be configured with a tip electrode. In some implementations, the tips of the connectors 140a, 140b may be insulated or may otherwise not provide a conductive path from connectors 140a, 140b to the receptacle. Stated another way, the tip electrode may be omitted. In addition, the connectors may be configured with any suitable number of pole/ring connections for supporting high or low current and with or without a tip electrode.

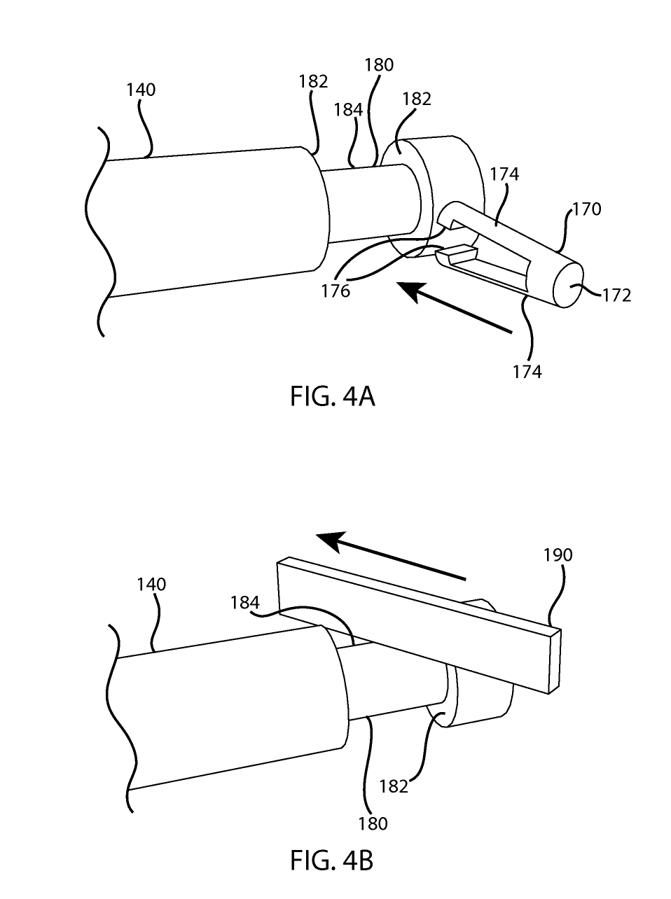

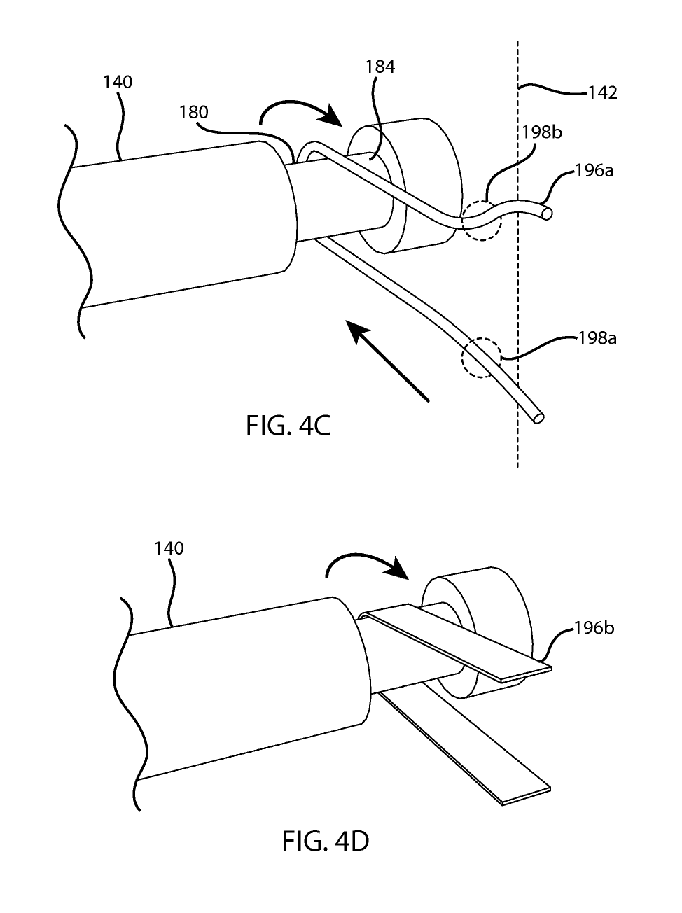

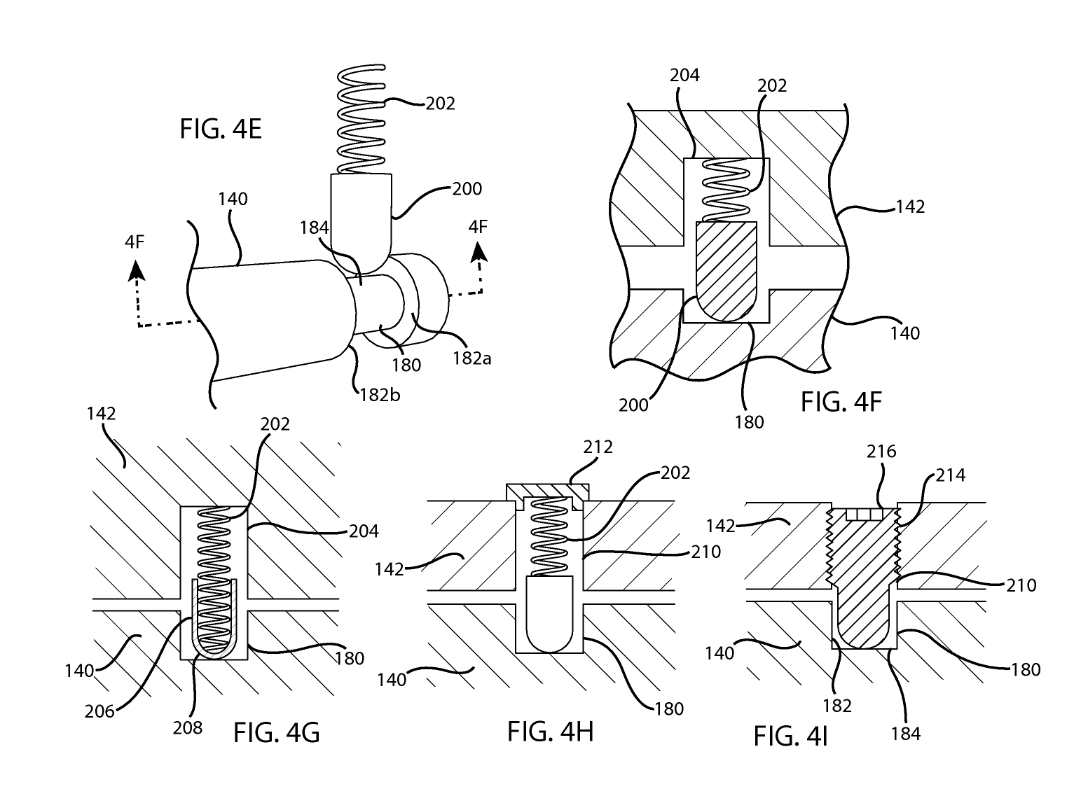

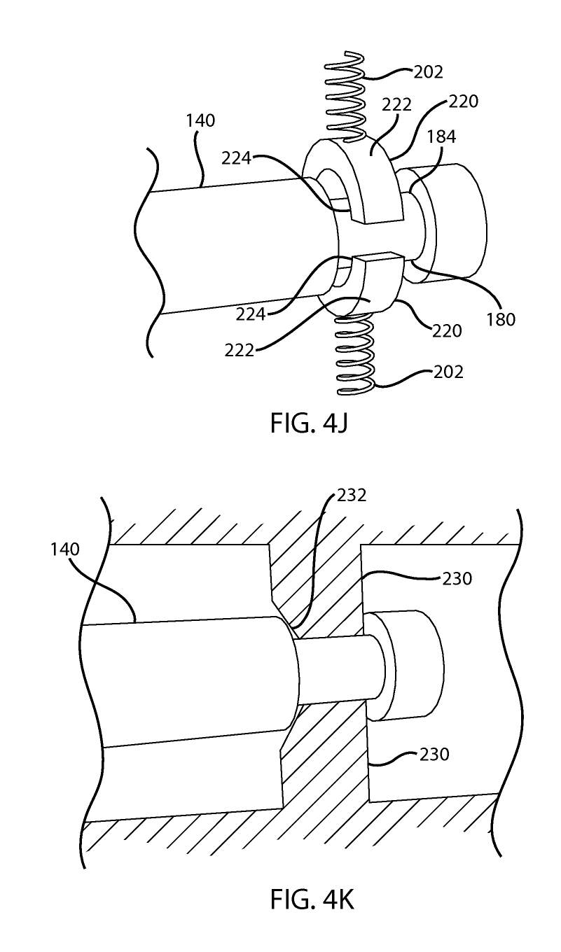

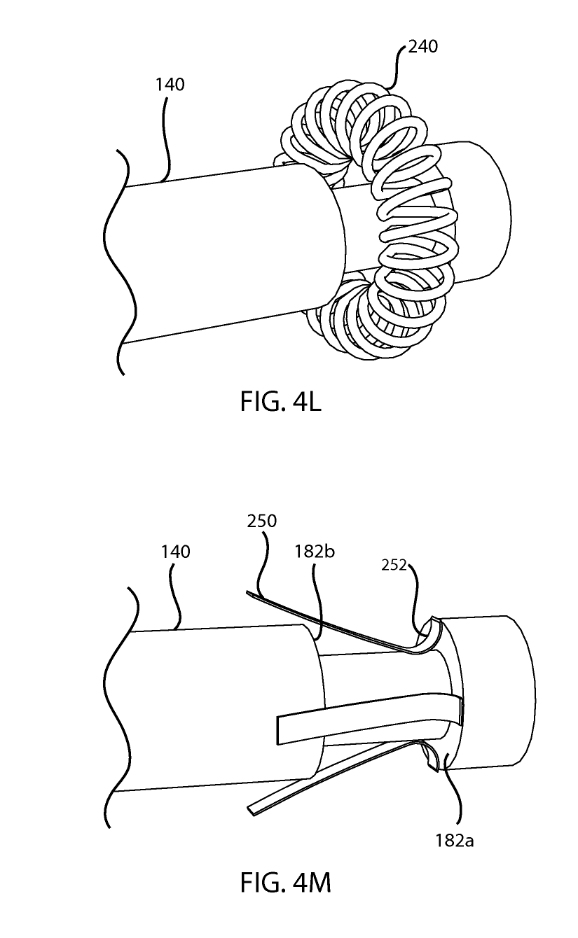

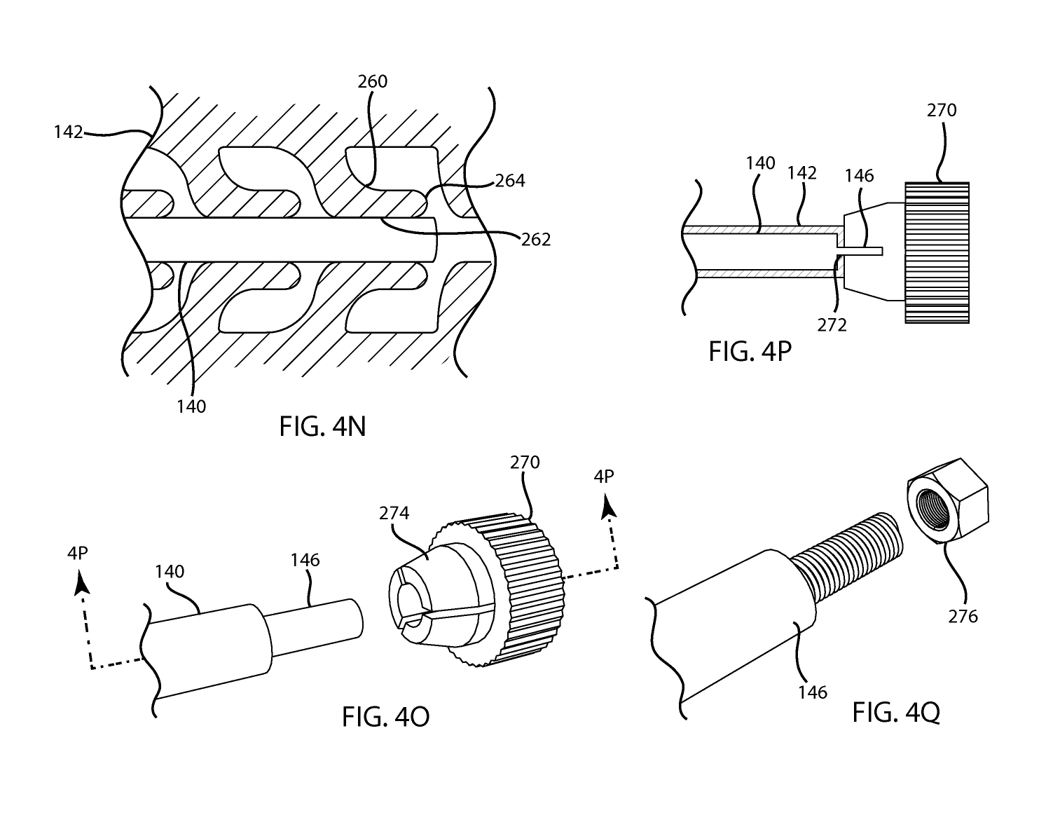

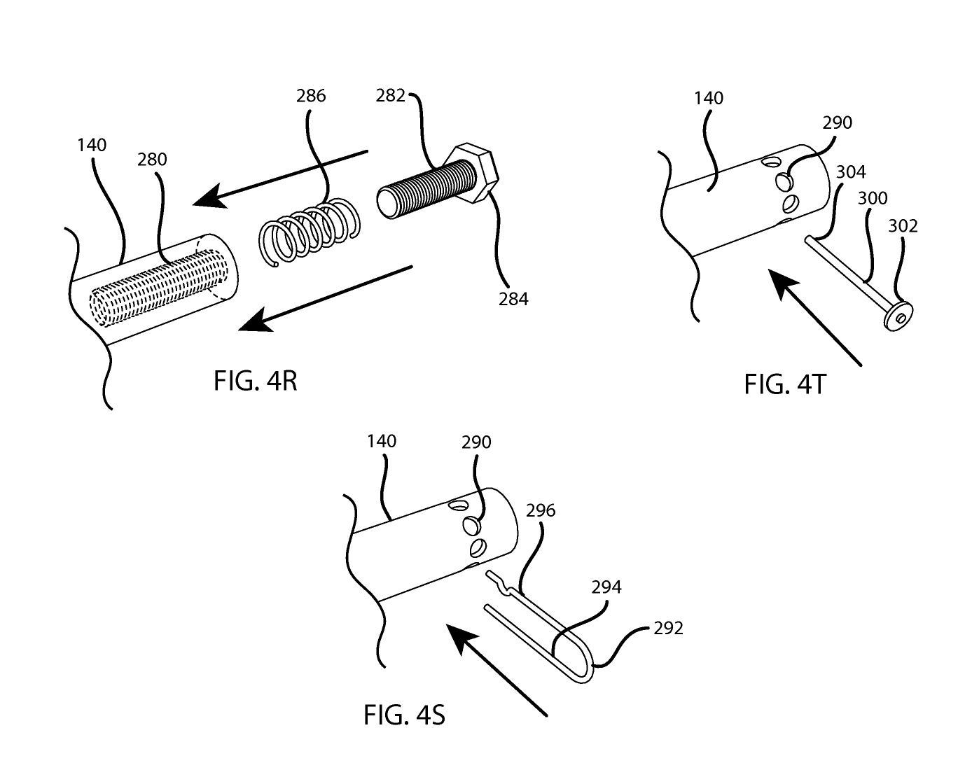

FIGS. 4A to 4T schematically illustrate various retention mechanisms for providing mechanical and/or electrical coupling between the various connectors and the mating receptacles. Depending on the way the retention mechanism is activated to engage the connector within the receptacle, the retention mechanism may be a user-actuated (or manual) retention mechanism or an automatic retention mechanism. If the retention mechanism includes a user manually placing a retention member or part to engage the connector, the retention mechanism may be referred to as a user-actuated retention mechanism. If the retention mechanism may automatically engage the connector once the connector is inserted into the receptacle, the retention mechanism may be referred to as an automatic retention mechanism. If a user-actuated retention mechanism is used, the user may first remove a casing of the header 104 or other component having the receptacle to place the retention mechanism in engagement with the connector. Once the retention mechanism is in place, the user may replace the casing portion of the header 104 to hermetically seal the components within the header 104 or other component.

With reference to FIG. 4A, a first example of a user-actuated retention mechanism for securing the connector 140 received within a receptacle 142 configured in the header 104 or other component will be described. The retention mechanism may include a retention clip 170. The retention clip 170 may include a clip head 172 and two clip arms 174 extending from the clip head 172. The clip head 172 and the clip arms 174 may collectively define a U shape. Each of the clip arms 174 may include a protrusion 176 at the free end of the arm 174 extending towards the other arm 174. To engage the connector 140 received within the receptacle 142, a user may place the retention clip 170 through a bore or aperture formed at the receptacle 142 to allow the arms 174 of the clip 170 to engage a circumferential groove 180 or recess formed at the connector 140.

In continuing reference to FIG. 4A, the connector 140 may include a circumferential groove 180 or recess for engaging the retention clip 170. The groove 180 may be formed near the distal end, the proximal end, or at any suitable location of the connector 140. Although only one groove 180 is shown, the connector 140 may include more than one groove 180 formed at any suitable location spaced apart along the longitudinal dimension of the connector 140. The circumferential groove 180 may include two side wall portions 182 and a base portion 184 joining the two side wall portions 182. Each of the side walls 182 and the base portion 184 of the groove 180 may define a corner or a transition area therebetween with a sharp, smooth, curved, arcuate, or rounded appearance. The side walls 182 of the groove 180 may define substantially planar wall surfaces. The base portion 184 of the groove 180 may define a general cylindrical surface. In some examples, the side walls 182 and/or base portion 184 of the groove 180 may define other surface features, such as a concave surface, a convex surface, an angled surface, and/or a combination thereof.

The spacing between the two arms 174 of the retention clip 170 may be configured substantially the same as or less than the diameter of the base portion 184 of the groove 180 so as to closely engage the surface of the base portion 184 of the groove 180. The spacing between the protrusions 176 may be configured to be less than the diameter of the base portion 184 so that when the clip 170 engages the groove 180, the protrusions 176 may prevent the clip 170 from accidentally backing out. Due to such spacing configuration, the clip arms 174 may undergo some elastic deformation when the retention clip 170 is being inserted to engage the groove 180. The clip 170 may be formed using a plastic or rubber material or any suitable material having an insulation coating/protection if the clip 170 is not required to form electrical connection between the receptacle and the connector 140. The clip 170 may be formed using any suitable metal or conductive material, if needed for electrical connection. Once the clip 170 engages the groove 180 of the connector 140, the connector 140 may be secured within the receptacle and may be prevented from accidentally backing out of the receptacle.

In some examples, the opposing surfaces of the clip arms 174 that engage the base portion 184 of the groove 180 may be substantially flat for easy manufacturing. In some examples, the opposing surfaces of the clip arms 174 may define two concaved surfaces that substantially conform to the outer surface of the base portion 184 of the groove 180. In some examples, the free ends of the clip arms 174 may each be formed with a curved/convex lip portion to facilitate the insertion of the retention clip 170 along the surface of the base portion 184 of the groove 180. In some examples, the clip arms 174 may include an arm width less than the distance between the two side walls 182 of the groove 180 for easy insertion. In some examples, the width dimensions of the arms 174 may be configured to be substantially similar to the distance between the two side walls 182 of the groove 180 to prevent longitudinal movement of the connector 140. In any event, when the clip 170 engages the groove 180 of the connector 140, the retention clip 170 may prevent the connector 140 from accidentally backing out of the receptacle.

The retention clip 170 may be configured with a suitable length such that when the clip 170 engages the connector 140, at least the clip head 172 may still engage the bore formed in the receptacle of the header 104 or other component. The clip head 172 may be configured with a diameter substantially the same as the diameter of the bore so that the clip head 172 may be held in place by a friction-fit. In some examples, a cap may be used to seal the bore and retain the clip within the bore. In some examples, the clip 170 may simply be held in engagement with the connector 140 by the protrusions 176 at the free end of the arm 174.

With reference to FIG. 4B, another example of a user-actuated retention mechanism will be described. The connector 140 may be formed with one or more circumferential grooves/recesses 180 along the longitudinal dimension of the connector 140 similar to that as described with reference to FIG. 4A. After the connector 140 has been inserted into the receptacle within the header 104, an elongated retention key 190 or wedge may be positioned through a bore formed in the receptacle of the header 104 and engage the groove 180 of the connector 140 to prevent longitudinal movement of the connector 140 within the receptacle.

Although the retention key 190 is shown having a rectangular cross section, it is contemplated that the retention key 190 may have cross sections of any suitable shape, such as square, rectangular, triangular, tubular, circular, semi-circular, I-beam, U-shape, and so on. The surface of the retention key 190 that engages the base portion 184 of the groove 180 may be substantially planar, concave, convex, or a combination thereof. The retention key 190 may be configured with a cross section substantially the same as the cross section of the bore formed at the receptacle and at least a portion of the retention key 190 may be held in place by a friction-fit therebetween. In some examples, a cap or stopper, either formed as a separate part from the retention key 190 or formed as an integral part of the retention key 190, may be used for preventing the retention key 190 from backing out.

The retention key 190 may include a height similar to or less than the height of the side walls 182 of the groove 180 (i.e., the depth of the groove 180). The retention key 190 may include a height greater than the depth of the groove 180. The receptacle may include a recess or notch formed to accommodate the height of the retention key 190. This way, the retention key 190 may further limit the relative longitudinal movement between the connector 140 and the receptacle and secure the connector 140 within the receptacle. The retention key 190 may include a width substantially the same as or similar to the width of the groove 180 of the connector 140 to facilitate longitudinal alignment between the connector 140 and the receptacle and prevent any longitudinal movement of the connector 140 within the receptacle. In some examples, the retention key 190 may be configured with a width less than that of the groove 180 of the connector 140 for easy insertion.

With reference to FIGS. 4C and 4D, the connector 140, formed with one or more circumferential grooves 180, may be secured within the receptacle using a retention member such as wires 196a or bands 196b. Once the connector 140 is positioned within the receptacle, a retention member such as a wire 196a or band 196b may be positioned into the receptacle through an inlet aperture or bore 198a formed in the receptacle 142 of the header 104 to engage the connector 140. The gap or space between the connector 140 and the receptacle 142, defined in part by the groove 180 of the connector 140, may form a channel that may guide the wire 196a or band 196b to pass around the base portion 184 of the groove 180. The receptacle 142 may be formed with an outlet aperture or bore 198b to further guide the wire 196a or band 196b out of the receptacle 142.

Although one loop of a retention member such as wire 196a or band 196b is shown engaging the groove 180 of the connector 140, two or more loops of the wire 196a or band 196b may be formed by passing the wire 196a or band 196b multiple times through the inlet 198a and the outlet 198b of receptacle 142 and the guiding channel formed by the connector 140 and the receptacle 142. The ends of the wire 196a or band 196b may be held in place by tying the ends together. Alternatively, the ends may be tied to a cap that may include protrusions engaging the inlet 198a and the outlet 198b formed at the receptacle 142.

The wire 196a used for engaging and securing the connector 140 within the receptacle 142 may include wires such as suture wires that may be formed of any suitable material. The band 196b may be configured with any appropriate band width that may fit in the groove of 180 of the connector. The band 196b may be elastic, stretchable, or non-stretchable and may be formed of any suitable material. Because the receptacle 142, the connector 140, and the wire 196a or band 196b may be enclosed in the hermetically sealed header 104, and may not be in direct contact with human tissues, other non-surgical wires, bands, threads may be used as long as it may provide the mechanical strength needed.

With reference to FIGS. 4E and 4F, the connector 140 may be secured within the receptacle 142 using a pin member configured to engage a circumferential groove 180 of the connector 140. The pin member 200 may be held against the base portion 184 of the groove 180 by a spring member 202, such as a coil spring. Specifically, one end of the pin member 200 may define an exteriorly convex surface configured to engage the connector 140, and the other end of the pin member 200 may be operably coupled to one end of the spring member 202. The other end of the spring member 202 may be further operably coupled to the bottom of a recess or a notch 204 formed in the interior surface of the receptacle 142. The spring member 202 and the pin member 200 may be configured such that when the spring member 202 is not in compression, the spring member 202 and a portion of the pin member 200 may be received within the recess 204 of the receptacle 142, and a portion of the pin member 200 may protrude from the interior surface of the receptacle 142. The spring member 202 and the pin member 200 may be further configured such that when the spring member 202 is compressed, the spring member 202 and the entirety or a substantial portion of the pin member 200 may be received within the recess of the receptacle 142.

When the connector 140 is being inserted into the receptacle 142, the tip or the leading portion of the connector 140, which may define an exteriorly convex or slanted surface, may push the pin member 200 to retract into the recess 204 as the connector 140 proceeds. As the connector 140 is fully inserted into the receptacle 142, the groove 180 of the connector 140 may align with the recess 204 of the receptacle 142 and a portion of the pin member 200 may be forced by the spring member 202 to protrude into the groove 180 of the connector 140 to engage the base portion 184 of the groove 180. The pin member 200 may be configured with a width or diameter substantially the same as or similar to the width of the groove 180 (or the distance between the side walls 182a, 182b of the groove 180) such that when the pin member 200 engages the groove 180, the pin member 200 and the side walls 182 of the groove 180 may limit the longitudinal movement of the connector 140 and/or prevent the connector 140 from backing out of (or being disconnected from) the receptacle 142. Since the pin member 200 and the spring member 202 may automatically engage the groove 180 of the connector 140 as the connector 140 is fully inserted into the receptacle 142, the retention mechanism may be referred to as an automatic retention mechanism.

In some examples, the receptacle 142 may be configured with more than one spring loaded pin member 200 each configured to engage a circumferential groove 180 of the connector 140. To facilitate the insertion of the connector 140 into the receptacle 142, the trailing side wall 182b of each circumferential groove 180 with respect to the insertion direction of the connector 140 and an adjacent portion of the exterior surface of the connector 140 may define an exteriorly convex or slanted surface. The leading side wall 182b of each circumferential groove 180 and an adjacent portion of the exterior surface of the connector 140 may define an angle equal to or less than 90 degree for retention purpose.

With reference to FIG. 4G, for weight reduction or size considerations, the pin member may be configured as a hollow cylindrical (or tubular) shell 206 having a capped end 208. The capped end 208 may define an exteriorly convex surface or a tapered surface configured to engage the groove 180 of the connector 140. A portion of the spring member 202 may be received within the shell 206 and operably coupled to the interior surface of the end cap 208. The capped shell 206 may function in a manner similar to the pin member 200 as described above. The capped shell 206 may fully retract and be received within the recess 204 of the receptacle 142 as the connector 140 is being inserted into the receptacle 142. When the connector 140 is fully inserted, the capped shell 206 may protrude and be forced by the spring member 202 to engage the groove 180 of the connector 140.

With reference to FIG. 4H, instead of forming a recess 204 within the receptacle 142 for receiving the spring member 202 and the pin or shell member 200, 206, the receptacle 142 may be formed with a through hole 210. A closure member 212 may be arranged at the exterior opening of the through hole 210. The closure member 212 may be configured to operably join the spring member 202, which may be further joined to the pin or capped shell member 206 for engaging the groove 180 of the connector 140. In this instance, the closure member 212, the spring member 202, and the pin or capped shell member 206 may define a structure similar to a pogo pin. Forming a through hole 206 at the receptacle 142 may allow the retention mechanism to be removed by removing the closure member 212, as well as the spring member 202 and the pin or shell member 200, 206 joined thereto, from the exterior opening of the through hole 212. Removal of the retention mechanism may allow the connector 140 to be removed from the receptacle 142 and be replaced with a different connector 140.

In some examples, the closure member 212, as well as the spring member 202 and the pin or capped shell member 206 joined thereto, may be provided at the receptacle 142 during assembly of the header 104. As the connector 140 is inserted into the receptacle 142, the pin or capped shell member 206 may automatically engage the connector 140 by the force of the spring member 202. In some examples, the closure member 212, the spring member 202, and the pin or capped shell member 206 may be placed by a user, such as a surgeon during an operation, after the connector 140 has been fully inserted into the receptacle 142. In this case, the closure member 212, the spring member 202, and the pin or capped shell member 206 may or may not be operably joined to each other. They may contact each other and be held within the space defined by the through hole 210 of the receptacle 142 and the groove 180 of the connector 140 by the engagement between the closure member 212 and the exterior opening of the through hole 210 of the receptacle 142. Since the closure member 212, the spring member 202, and the pin or capped shell member 206 are placed by a user for engaging the connector 140, the retention mechanism in this instance may be referred to as a user-actuated (or manual) retention mechanism.

With reference to FIG. 4I, the through hole 210 may be provided with threading 214. The closure member 212 may include a fastener, such as a screw, that engages the treading 214. In further examples, the closure member 212, the spring member 202, and the pin or shell member 200, 206 may be replaced with a single fastener member, such as a set screw 216. The tip portion of the set screw 216 may be held against the base portion 184 of the groove 180 of the connector 140 when the set screw 216 engages the threading 214 in the through hole 210 of the receptacle 142. The pressure applied by the tip portion of the set screw 216 may limit the longitudinal movement of the connector 140 within the receptacle 142. The side walls 182 of the groove 180 may or may not be in direct contact with side portions of the set screw 216. In either case, the side walls 182 of the groove 180 may prevent the connector 140 from being accidentally disconnected from the receptacle 142 by holding the set screw 216 within the grooved portion of the connector 140.

With reference to FIG. 4J, the retention mechanism may include a pair of spring loaded clamp members 220 positioned at opposite sides of the connector 140. Each clamp member 220 may define a C or other suitable shape. The convex surface 222 of each C-shaped clamp member 220 may be operably coupled to a spring member 202. When the connector 140 is inserted into the receptacle 142, the concave surface 224 of each of the C-shaped clamps 220, biased by the spring member 202, may engage a surface portion of the base portion 184 of the circumferential groove 180 of the connector 140. The clamps 220 may limit the longitudinal movement of the connector 140 within the receptacle 142 and/or prevent the connector 140 from backing out of (or being disconnected from) the receptacle 142. The receptacle 142 may include a pair of opposing recesses or notches 204 or a pair of opposing through holes 210, similar to the examples described with reference to FIGS. 4E-4H. The spring members 202 and/or portions of the clamp members 220 may be received within and joined to opposing recesses 204 of the receptacle 142. In some examples, the spring members 202 and/or portions of the clamp members 220 may be received within the through holes 210 of the receptacle 142 and retained therein by closure members 212 arranged at the exterior openings of the through holes 210. The spring loaded clamp members 220 may automatically engage the connector 140 as the connector 140 is inserted into the receptacle 142. Alternatively, the spring loaded clamp members 220 may be positioned through the through holes 210 of the receptacle 142 by a user after the connector 140 is inserted into the receptacle 142 to engage the connector 140.

With reference to FIG. 4K, the retention mechanism may include a plurality of protrusions 230 spaced at angular intervals or an annular protrusion 230 extending from the interior surface of the receptacle 142. The annular protrusion or the plurality of protrusions 230 may define a center opening smaller than the diameter of the connector 140. The opening may be smaller than or similar to the diameter of the groove 180 of the connector 140. The protrusion(s) 230 may be formed by an elastic material, such as a prolapsing silicone. As the connector 140 is being inserted into the receptacle 142, the protrusion(s) 230 may experience elastic deformation to allow the non-grooved portion of the connector 140 to pass through. It should be understood that a lead in chamfer 232 would help facilitate the insertion and hinder the retraction of the connector 140. The lead in chamfer 232 may be configured at the surface of the protrusions 230 facing the opening of the receptacle 142, through which the connector 140 is inserted. Alternatively, a sloped surface, similar to the lead in chamfer at the protrusions 230, may be configured at the trailing side wall and/or the tip portion of the connector 140. When the connector 140 is fully inserted into the receptacle 142, the protrusion(s) 230 may restore partially or completely its shape to engage the groove 180 of the connector 140.

Although the elastic retention mechanism is described as protrusion(s) 230 from the interior surface of the receptacle 142, it should be understood that the elastic retention mechanism may include an annular ring member arranged at the groove 180 of connector 140. The annular ring member may mechanically retain the connector 140 within the receptacle 142 by engaging a recess formed in the receptacle 142 or by a friction fit between the annular ring member and the interior surface of the receptacle 142. Any suitable configuration or geometry for protrusion(s) 230 may be used.

With reference to FIG. 4L, the retention mechanism may include a spring member 240, such as a coil member, provided around the groove 180 of the connector 140. When the connector 140 is inserted into the receptacle 142, the coil member 240 may provide mechanical retention to the connector 140 within the receptacle 142 by engaging a recess formed in the receptacle 142 or by a friction fit between the coil and the interior surface of the receptacle 142. In this example, the groove 180 may be formed at a pole connection (or ring connection) 144a, 144b of the connector 140, and the coil 240 may be made of a conductive material. The coil 240 may mechanically retain the connector 140 as well as electrically connecting the connector 140 to the receptacle 142.

With reference to FIG. 4M, another example of the retention mechanism may include a compression mechanism provided around the groove 180 of the connector 140. The compression mechanism may include one or more leaf springs 250 arranged in spaced angular intervals around the groove 180 of the connector 140. One end of each of the leaf springs 250 may be operably positioned within the grooved portion of the connector 140 at a location adjacent to the leading side wall 182a of the groove 180 with respect to the insertion direction of the connector 140. The leaf springs 250 may be oriented to extend toward the trailing side wall 182b of the groove 180 and extend away from the outer surface of the connector 140, or otherwise as desired (including being arranged partially or wholly transversely) to the groove 180. Such orientation of the plurality of the leaf springs 250 may allow the travel of the connector 140 in the direction when it is being inserted into the receptacle 142 and may hinder or prevent the travel of the connector 140 in the opposite direction. Therefore, the plurality of the leaf springs 250 may substantially increase the force or resistance for the connector 140 to back out of the receptacle 142, thereby ensuring the connection between the connector 140 and the receptacle 142.

In some implementations, the plurality of the leaf spring 250 may each be operably joined to the inner surface of the receptacle 142 and arranged in spaced angular intervals. In this case, the leaf springs 250 may each extend toward the center of the receptacle 142 and away from the opening of the receptacle 142 from which the connector 140 may be inserted. As such, the free ends of the leaf springs 250 may collectively define an opening less than the diameter of the non-grooved portion of the connector 140 or even less than the diameter of the grooved portion of the connector 140. Such configuration of the leaf springs 250 may allow the connector 140 to be inserted into the receptacle 142 and limit the backward movement of the connector 140. When the connector 140 is fully inserted into the receptacle 142, the free ends of the leaf springs 250 may engage the groove 180 of the connector 140. In some implementations, the free ends of the leaf springs 250 may engage the leading side wall 182a and press against the side wall 182a, which may substantially increase the force or resistance for the connector 140 to back out of the receptacle 142.

In some implementations, the free ends of the leaf springs 250 may each be configured with a bent portion 252. Such bent portions 252 may allow for the removal or disengagement of the connector 140. When a pulling force is applied to the connector 140, the pulling force being sufficient to overcome the resistance created by the engagement between the leaf springs 250 and the leading side wall 182a, the bent portion 252 may slide along the leading side wall 182a out of the groove 180 thereby disengaging the leading side wall 182a of the groove so that the connector 140 may be removed or disengaged from the receptacle 142 when needed.

With reference to FIGS. 4N to 4T, further examples of retention mechanisms will the described. The connector 140 using these retention mechanisms may or may not be formed with a groove 180. With reference to FIG. 4N, the interior surface of the receptacle 142 may be provided with one or more elastic sealing members 260. Each of the elastic sealing members 260 may be defined by an annular protrusion from the interior surface of the receptacle 142. As the connector 140 is being inserted into the receptacle 142, the exterior surface of the connector 140 may contact the elastic sealing members 260. The friction between the exterior surface of the connector 140 and the elastic sealing members 260 may cause the sealing members 260 to bend and to form an increased surface contact area 262 with the exterior surface of the connector 140. The free ends 264 of the sealing members 260 may point in the insertion direction of the connector 140. The increased surface contact area 262 and the orientation of the free ends 264 of the sealing members 260 may hold the connector 140 within the receptacle 142 by friction and prevent the connector 140 from moving in the opposite direction, thereby securing the connector 140 within the receptacle 142.

In some implementations, the surface 266 of each of elastic sealing members 260 that faces the opening of the receptacle 142, through which the connector 140 may be inserted, may be at least partially oriented at an angle, such as at an obtuse angle, with respect to the inner surface of the receptacle 142. Such angled orientation may create an increased thickness of the elastic sealing members 260 near the inner surface of the receptacle 142 and result in an asymmetrical shape of the sealing members 260. The asymmetry of the sealing members 260 may allow for easy insertion of the connector 140 while simultaneously increasing the retention or withdrawal force.

With reference to FIGS. 4O and 4P, the retention mechanism may include a holding member 270, such as a collet, configured for holding a tip portion of the connector 140, such as the tip electrode 146 of the connector 140. The receptacle 142 may be configured with an opening 272 for the tip electrode 146 to pass through and extend outside the receptacle 142 when the connector 140 is fully inserted into the receptacle 142. A collet 270 may be used to clamp around the tip electrode 146. When the collet 270 securely holds the tip electrode 146, the collar 274 of the collet 270 holding the tip electrode 146 may abut the end of the receptacle 142 where the opening 272 is formed, thereby limiting or preventing relative longitudinal movement of the connector 140 within the receptacle 142 and securing the connection between the connector 140 and the receptacle 142. Alternately, the tip 146 of the connector 140 may be threaded and thus engage with a threaded nut 276 (FIG. 4Q) to pull in and secure the connector 140.

With reference to FIG. 4R, the connector 140 may be formed with internal threading 280. A bolt or screw fastener 282 may be positioned through an opening 272 formed at the receptacle 142 to engage the internal threading 280 of the connector 140. When the bolt or screw fastener 282 is engaged with the connector 140, the tip or end of the connector 140 may engage an interior surface of the end of the receptacle 142 where the opening 272 is formed, and the head 284 of the bolt or screw fastener 282 may engage the exterior surface of the end of the receptacle 142. This way, the connector 140 may be held by the bolt or screw fastener 282 against the end of receptacle 142 and may be prevented from backing out of the receptacle 142. In some examples, a spring member 286, such as a coil spring, may be sleeved on the bolt or screw fastener 282 and may be biased between the exterior surface of the end of the receptacle 142 and the head 284 of the bolt or screw fastener 282. Alternatively, the spring member 286 may be biased between the interior surface of the end of the receptacle 142 and the connector 140.

With reference to FIG. 4S, the connector 140 may be formed with one or more transverse through holes 290 arranged in spaced angular intervals. A pin, such as an R-shaped cotter pin or split pin 292, may be first positioned through an aperture formed in the receptacle to engage the connector 140. In particular, the straight arm 294 of a cotter pin 292 may be further positioned through one of the transverse through holes 290 of the connector 140 and the arm having the curved portion 296 of the cotter pin 292 may securely engage the outer surface of the connector 140. Forming multiple transverse through holes 290 at the connector 140 may facilitate alignment between one of the transverse through holes 290 of the connector 140 with the aperture formed at the receptacle, thereby facilitating the insertion of the cotter pin 292.

With reference to FIG. 4T, in some examples, a pin such as a straight pin 300 may be used. The pin 300 may have an enlarged head portion 302, or it may not, in which case it may be bent to provide for a secure attachment. The straight pin 300 may be positioned through the aperture at the receptacle and then through one of the transverse through holes 290 of the connector 140. The enlarged head portion 302 of the pin 300 may securely engage the aperture of the receptacle and may prevent the pin 300 from retracting out of the connector 140 and/or the receptacle. In some examples, a cap may be provided at the aperture of the receptacle to enclose the pin 300 within the receptacle and the connector 140. In further examples, the end portion 304 of the pin 300 opposite to the enlarged head portion 302 may be bent to lock at the connector 140 after it is positioned through the transverse through hole 290 of the connector 140.

The various examples of retention mechanisms described above without limitation may be advantageously used separately in some instances, or in combination with one another in other instances. The retention mechanisms may provide only mechanical retention for the connector within the receptacle in some instances, or may provide electrical connection between the connector and the receptacle, in addition to mechanical retention for the connector within the receptacle in other instances. The components and materials used for forming the retention mechanism may be insulating or conductive, depending on the specific application.

Although only one pair of connector and receptacle is described herein as examples, the header may include more than one receptacle configured in a similar manner or differently for receiving therein a connector for connecting to any suitable implantable devices, power systems, or any appropriate components the implantable system may include. Although only one groove is shown for each connector, the various connectors may include more than one groove formed at any suitable location spaced apart along the longitudinal dimension of the connector.

Although the connection mechanisms are described herein for coupling a cable end to the header of the implantable converter or control system, it should be noted that the various connection mechanisms may be used for electrically and mechanically coupling a cable end to any one of the implantable devices or systems. The various connection mechanisms may also be used to electrically and mechanically couple two cable ends. The various retention mechanisms may be used for any of the connection mechanisms when suitable to ensure the connection between the connector and the receptacle.

Although the connection mechanisms and the retention mechanisms may be used for continuous high power delivery, they may also be used for intermittent high power delivery or continuous or intermittent low power delivery.

There are many advantages of the connection mechanisms and the retention mechanisms described herein. Since the implantable device, such as a mechanical circulatory device or a heart pump, may be connected to the implantable control system using only one connector, misconnection between the motor of the heart pump and the converter or controller may be prevented. Therefore, the motor of the heart pump may be prevented from starting in the wrong direction, which could be dangerous and life-threatening for the patient receiving the implantable device. In addition, the connectors used for connecting the implantable TETS to the converter or controller may be physically different from the connectors used for the mechanical circulatory device. Therefore, misconnections between the device and the TETS coil to the converter or controller may be prevented. The multi- or single-pole connectors described herein may be configured with relatively small sizes and can be easily inserted into the receptacles of converter or controller during surgery. Moreover, the retention mechanisms may secure the connection between the respective connectors and receptacles, thereby enhancing the reliability of the entire implantable system within the body of the patient receiving such system. Furthermore, since the connection mechanisms may be hermetically sealed and electrically isolated within the header of a converter or controller, the connection mechanisms may be protected from the environment. The connection mechanisms and/or the retention mechanisms may allow for continuous high current connections for continuous high power delivery, which may not be accomplished by conventional connectors.

It should be noted that all directional and/or dimensional references (e.g., upper, lower, upward, downward, left, right, leftward, rightward, top, bottom, above, below, front, back, rear, forward, backward, rearward, inner, outer, inward, outward, vertical, horizontal, clockwise, counterclockwise, length, width, height, depth, and relative orientation) are only used for identification purposes to aid the reader's understanding of the implementations of the disclosed invention(s), and do not create limitations, particularly as to the position, orientation, use relative size or geometry of the invention(s) unless specifically set forth in the claims.

Connection references (e.g., attached, coupled, connected, joined, and the like) are to be construed broadly and may include intermediate members between a connection of elements and relative movement between elements. As such, connection references do not necessarily infer that two elements are directly connected and in a fixed relation to each other.

In some instances, components are described with reference to “ends” having a particular characteristic and/or being connected with another part. However, those skilled in the art will recognize that the disclosed invention(s) is not limited to components that terminate immediately beyond their points of connection with other parts. Thus, the term “end” should be interpreted broadly, in a manner that includes areas adjacent, rearward, forward of, or otherwise near the terminus of a particular element, link, component, part, member or the like. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made that are within the scope of the appended claims.

1. A connection mechanism for an implantable system, comprising:
a connector;
a receptacle configured for receiving therein the connector to establish high power or high current electrical communication between the connector and the receptacle; and
a retention mechanism for mechanically and/or electrically engaging the connector within the receptacle.

2. The connection mechanism of claim 1, wherein
the implantable system further comprises an implantable device, an implantable converter, and an implantable power system; and
the connection mechanism is configured to establish high power or high current electrical communication between the converter and one of the implantable device or the power system.

3. The connection mechanism of claim 2, wherein the power system comprises an implantable transcutaneous energy transfer system.

4. The connection mechanism of claim 2, wherein the implantable device comprises a mechanical circulatory device.

5. The connection mechanism of claim 2, wherein the receptacle is at least partially hermetically sealed within a header portion of the converter.

6. The connection mechanism of claim 1, wherein
the connector comprises a circumferential groove having two annular side wall portions and a cylindrical base portion between the side wall portions; and
the retention mechanism is configured to engage circumferential the groove of the connector by engaging at least one of the side wall portions or the base portion.

7. The connection mechanism of claim 6, wherein the retention mechanism comprises a retention clip including two arms each configured to engage a surface portion of the groove of the connector and having a protrusion formed at the free end thereof.

8. The connection mechanism of claim 6, wherein the retention mechanism comprises a retention key oriented perpendicular to the longitudinal extension of connector.

9. The connection mechanism of claim 6, wherein the retention mechanism comprises one of a wire or a band looped around the groove of the connector.

10. The connection mechanism of claim 6, wherein the retention mechanism comprises a compression biased mechanism.

11. The connection mechanism of claim 10, wherein the compression biased mechanism is biased against the groove of the connector by a spring member.

12. The connection mechanism of claim 10, wherein the compression biased mechanism at least partially retracts into a recess formed within the receptacle when the connector is being inserted into the receptacle.

13. The connection mechanism of claim 10, wherein the compression biased mechanism comprises one of a pin member, a shell member, or a pair of clamp members operably coupled to a spring member.

14. The connection mechanism of claim 6, wherein the retention mechanism comprises an elastic protrusion extending from the receptacle, the elastic protrusion configured to engage the groove of the connector by being at least partially received in the groove of the connector.

15. The connection mechanism of claim 6, wherein the retention mechanism may include a spring/compression member configured around the groove of the connector.

16. The connection mechanism of claim 15, wherein the spring/compression member comprises one of at least one leaf spring.

17. The connection mechanism of claim 1, wherein the retention mechanism comprises a plurality of elastomeric sealing members arranged at the receptacle, the elastomeric sealing members configured to engage the connector by friction or surface contact between the elastomeric sealing member and connector.

18. The connection mechanism of claim 1, wherein the retention mechanism comprises a fastener configured to engage a tip portion of the connector through an aperture formed at an end portion of the receptacle.

19. The connection mechanism of claim 18, wherein the fastener comprises one of a collet, a screw, or a bolt.

20. The connection mechanism of claim 18, wherein the fastener and/or the tip portion of the connector may be biased against the end portion of the receptacle.

21. The connection mechanism of claim 1, wherein the retention mechanism comprises a cotter pin configured to engage at least one through hole formed at the connector.
